SEQUENCE LISTING (14 sequences)

SEQ ID NO: 1 - DNA, 20 bp, Artificial
  5′ Primer for polymerase chain reaction detect albumin gene
aaacctcttgtggaagagcc

SEQ ID NO: 2 - DNA, 20 bp, Artificial
  3′ Primer for polymerase chain reaction to detect albumin gene
caaagcaggtctccttatcg

SEQ ID NO: 3 - DNA, 20 bp, Artificial
  5′ Primer for polymerase chain reaction detect ASGPR gene
taggagccaagctggagaaa

SEQ ID NO: 4 - DNA, 20 bp, Artificial
  3′ Primer for polymerase chain reaction detect ASGPR gene
acctgcaggcagaagtcatc

SEQ ID NO: 5 - DNA, 20 bp, Artificial
  5′ Primer for polymerase chain reaction to detect bilirubin-UGT gene
atgacccgtgcctttatcac

SEQ ID NO: 6 - DNA, 20 bp, Artificial
  3′ Primer for polymerase chain reaction to detect bilirubin-UGT gene
tcttggatttgtgggctttc

SEQ ID NO: 7 - DNA, 20 bp, Artificial
  5′ Primer for polymerase chain reaction to detect GST-PI gene
gccctacaccgtggtctatt

SEQ ID NO: 8 - DNA, 20 bp, Artificial
  3′ Primer for polymerase chain reaction to detect GST-PI gene
ggctaggacctcatggatca

SEQ ID NO: 9 - DNA, 20 bp, Artificial
  5′ Primer for polymerase chain reaction to detect GS gene
atgctggagtcaagattgcg

SEQ ID NO: 10 - DNA, 20 bp, Artificial
  3′ Primer polymerase chain reaction to detect GS gene
tcattgagaagacacgtgcg

SEQ ID NO: 11 - DNA, 20 bp, Artificial
  5′ Primer for polymerase chain reaction to detect HBCF-X gene
gtgcatggaagagacctgct

SEQ ID NO: 12 - DNA, 20 bp, Artificial
  3′ Primer for polymerase chain reaction to detect HBCF-X gene
gaagtcaagcaggtcgaagg

SEQ ID NO: 13 - DNA, 18 bp, Artificial
  5′ Primer for polymerase chain reaction to detect SV40T gene
caggcatagagtgtctgc

SEQ ID NO: 14 - DNA, 20 bp, Artificial
  3′ Primer for polymerase chain reaction to detect SV40T gene
caacagcctgttggcatatg

(19) United States
(12) Patent Application Publication
(10) Pub. No.: US 2009/0148424 A1
(43) Pub. Date: Jun. 11, 2009

(54) Reversibly immortalized mammalian liver cells and use thereof

(76) Inventors: Naoya Kobayashi, Okayama (JP); Noriaki Tanaka, Okayama (JP); Michinori Kohara, Tokyo (JP)

Correspondence Address: THE NATH LAW GROUP, 112 South West Street, Alexandria, VA, 22314, US

(21) Appl. No.: 10/594,544

(22) PCT Filed: Mar. 31, 2004
(86) PCT No.: PCT/JP2004/004699

§ 371 Date: Oct. 17, 2007

(51) Int. Cl. A61K 35/12; C12N 5/06; C12N 5/08; A01N 1/02; C12N 15/00

(52) U.S. Cl. 424/93.21; 435/325; 435/370; 435/1.1; 435/320.1

(57) ABSTRACT

The present invention provides a reversibly immortalized mammalian liver cell line, especially CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) comprising an immortalizing gene interposed between a pair of site-specific recombination sequences and a suicide gene in the outside of the pair of site-specific recombination sequences, characterized in that the suicide gene can exhibit its function after excision of the pair of site-specific recombination sequences, or passage cell line thereof; a mammalian liver cell obtained by excising the immortalizing gene from the reversibly immortalized mammalian liver cell line or passage cell line thereof; and use of these cells. By utilizing the reversibly immortalized mammalian liver cell line of the present invention enables the obtainment of the number of the liver cells and utilization as materials for artificial liver reactors and cell preparations.

### TECHNICAL FIELD

The present invention relates to an immortalized mammalian liver cell capable of reversibly proliferating and containing a suicide gene even in the cell reverted. The present invention also relates to a bioartificial liver and a cell preparation using the liver cell.

### BACKGROUND ART

Currently, in Japan, two million hepatitis C infected patients (four million patients in the US) and about one and a half million hepatitis B patients (more than 120 million hepatitis B infected patients in China) are reported (Nikkei Science, 2000, February, Hepatitis Information Center). Annual fatality of hepatitis C complication is about 9,000 in Japan, and it is expected that the fatality will increase by threefold until 2010.

In view of these predictions, needs for hepatocyte transplantation and bioartificial organ for treating hepatic failure are high, and effective examples of human hepatocyte transplantation for metabolic hepatic disease are reported (see Strom S C, Fisher R A, Thompson M T, Sanyal A J, Cole P E, Ham J M, Posner M P., Transplantation (1997) 63: 559-569 and Fox I J, Roy Chowdhury J, Kaufman S S, et al., New Eng. J. Med. (1998) 338: 1422-1426).

Furthermore, a bioartificial liver using swine hepatocytes has been already in clinical trials on human in Europe, the US and China. However, the bioartificial liver causes apprehension about infectious diseases common to human and animal such as swine endogenous retrovirus infection, resulting in difficulty in future development.

Healthy human cells are ideal as a source for the cell transplantation and for the bioartificial liver. However, it is very difficult to obtain the cells due to the serious shortage of donors.

Accordingly, embryonic stem (ES) cells, stem cells and heterogeneous animal cells are investigated. However, utilization of the stem cells and precursor cells essentially involves difficulty in control due to pluripotency and active proliferation potency of the cells. Thus, there are reports on differentiation induction toward hepatocyte but researches on these cells is not adequate (see Rambhalta L, Chiu C P, Kundu P, et al., Cell Transplant. 2003; 12: 1-11; Schwartz R E, Reyes M, Koodie L, et al., J. Clin. Invest. 109; 1291-1302, 2002). Furthermore, for the heterogeneous animal cells, transplanted cells are not assured of being finally rejected. In human, a stable chimera state with the heterologous animal and accidental engraftment of HLA-mismatched homologous tumor are reported (see Gartner et al., N. Eng. J. Med., 335, 1494 (1996); K. Paradis et al., Science, 285, 1236, (1999)).

It is known that a cell line retaining an appropriate function for differentiation can be produced by transferring an oncogene to immortalize the cell (see Kobayashi N, et al., Transplantation 69:202-207, 2000). However, infusion of the immortalized cell line into a living body or use of the immortalized cell line in an extracorporeal circulation assist device such as bioartificial liver, there is a possibility that the patient may be exposed to unexpected risk of malignant alteration.

A reversibly immortalized cell which is immortalized by transferring an immortalizing gene and from which the immortalizing gene can been excised after proliferation of the cell is also investigated (see Westerman K A, Leboulch P., Proc. Natl. Acad. Sci., USA., Vol. 93, 8971-8976, (1996)). However, use of retroviral vectors involves a problem that tumors may be caused by the viral structural LTR and a defect that the number of transferred copy may be excess. Furthermore, even though the immortalizing gene is excised by using a site-specific recombinase, it is pointed out that there is a possibility of giving patients receiving medical treatment cause for anxiety, because there is a possibility that a genetically-altered cell remains in a host, and the cell exerts a bad influence upon the host (e.g. formation of tumors).

An object of the present invention is to solve the problems of the prior art, and to provide an immortalized mammalian liver cell which is used in place of a healthy human liver cell and can be proliferated in large quantity and whose livability can be regulated, resulting in higher safety, and a bioartificial liver and cell preparation using the same.

### DISCLOSURE OF INVENTION

As a result of an intensive study to solve the above problems, we have found that by transferring a vector encoding a suicide gene in the outside of an immortalizing gene interposed between a pair of site-specific recombination sequences to a mammalian liver cell, a safe liver cell line characterized in that it is an immortalized cell capable of reversibly proliferating and the suicide gene can exhibit its function after excision of the immortalizing gene, can be established, whereby the present invention has been completed.

Thus, the present invention relates to a reversibly immortalized mammalian liver cell line or the passage cell line thereof, containing an immortalizing gene interposed between a pair of site-specific recombination sequences and a suicide gene in the outside of the pair of site-specific recombination sequences, wherein the suicide gene can exhibit its function after excision of the pair of site-specific recombination sequences.

In the reversibly immortalized mammalian liver cell line, mammalian is preferably human.

The reversibly immortalized mammalian liver cell line preferably does not contain a promoter derived from virus.

The reversibly immortalized mammalian liver cell line is preferably CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657).

The present invention also relates to a mammalian liver cell obtainable by excising the immortalizing gene from the reversibly immortalized mammalian liver cell line or the passage cell line thereof (hereinafter referred to as “reverted cell”).

Furthermore, the present invention relates to a bioartificial liver comprising the reversibly immortalized mammalian liver cell line or the passage cell line thereof, or the reverted cells thereof.

The present invention also relates to a cell preparation comprising the reversibly immortalized mammalian liver cell line or the passage cell line thereof, or the reverted cells thereof.

The present invention relates to a non-viral vector comprising a non-vial promoter and encoding an immortalizing gene interposed between a pair of site-specific recombination sequences and a suicide gene in the outside of the pair of site-specific recombination sequences.

### BRIEF DESCRIPTION OF DRAWINGS

FIG. 1 is a schematic view showing a non-viral vector pYK1. Herein, ATG indicates starting codon, CAG indicates promoter sequence of CAG, LoxP indicates LoxP sequence, HygR indicates hygromycin-resistant gene, HSVTK indicates herpes simplex virus-thymidine kinase, EMCV IRES indicates encephalomyocarditis virus internal ribosome entry site, SV40T indicates simian virus 40 large T antigen gene, ZeoR indicates zeosin-resistant gene, and pA indicates poly-A signal.

FIG. 2 is a view showing an example of a method for preparing a non-viral vector pYK1. Herein, ATG indicates starting codon, CAG indicates promoter sequence of CAG, LoxP indicates LoxP sequence, HygR indicates hygromycin-resistant gene, HSVTK indicates herpes simplex virus-thymidine kinase, EMCV IRES indicates encephalomyocarditis virus internal ribosome entry site, SV40T indicates simian virus 40 large T antigen gene, ZeoR indicates zeosin-resistant gene, and pA indicates poly-A signal.

FIG. 3 is a view showing excision mechanism of a LoxP sequence by a DNA recombinase Cre. Herein, ATG indicates starting codon, LoxP indicates LoxP sequence, HygR indicates hygromycin-resistant gene, HSVTK indicates herpes simplex virus-thymidine kinase, EMCV IRES indicates encephalomyocarditis virus internal ribosome entry site, SV40T indicates simian virus 40 large T antigen gene, ZeoR indicates zeosin-resistant gene, and pA indicates poly-A signal.

FIG. 4 is a phase-contrast microscope image of a reversibly immortalized human hepatocyte CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657). Portion 1 denotes a nucleolus, Portion 2 denotes a large nucleus with some nucleoli and Portion 3 denotes intracellular granules.

FIG. 5 is a view showing an embodiment of a procedure of manufacturing an embodiment of a bioartificial liver reactor in accordance with the present invention. FIG. 5(a) is a view showing hollow fibers 6 arranged on a nonwoven cloth 5 provided with a backing material 4. The nonwoven cloth 5 provided with the backing material 4 is provided with a slit 7. FIG. 5(b) is a view showing a procedure of winding the sheet of FIG. 5(a) into a roll. FIG. 5(c) is an enlarged cross section of FIG. 5(b) taken on line X-X. FIG. 5(d) is a schematic view showing a bioartificial liver reactor 10 comprising a cylindrical container 9 with members 8 for preventing liquid leak provided at its both ends wherein a roll comprising the hollow fibers 6 and the nonwoven cloth 5 is incorporated. The reactor is provided with an inlet 11 for cells and an outlet 12 capable of sampling cells, and the slit 7 is arranged so as to communicate with the cell inlet 11.

FIG. 6(a) is a photograph showing the expressions of liver-specific genes and a SV40T gene in CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657). Lanes 1 to 7 indicate the expressions of albumin gene, asialoglycoprotein receptor (hereinafter refer to as ASGPR) gene, liver bilirubin-uridine phosphate glucuronocyl transferase (hereinafter refer to as bilirubin-UGT) gene, glutamine synthetase (hereinafter refer to as GS) gene, glutathione-S-transferase π (hereinafter refer to as GST-N) gene, human blood coagulation factor X (hereinafter refer to as HBCF-X) gene, and SV40T gene, respectively. M indicates marker.

FIG. 6(b) is a photograph showing the expressions of liver-specific genes and a SV40T gene in the reverted CYNK-1 cell. Lanes 1 to 7 indicate the expressions of albumin gene, ASGPR gene, bilirubin-UGT gene, GS gene, GST-N gene, HBCF-X gene, and SV40T gene, respectively. M indicates marker.

FIG. 7 is a graph showing a ganciclovir sensitivity of the reversibly immortalized human hepatocyte CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657).

FIG. 8 is a phase-contrast microscope image of the reversibly immortalized human hepatocyte CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) treated by ganciclovir. Dead cells 13 are observed.

FIG. 9 is a phase-contrast microscope image of the reverted CYNK-1 cells treated with ganciclovir. Dead cells 13 are observed.

FIG. 10 is a graph showing the survival curve of swines after administering D-galactosamine.

FIG. 11 is a scanning electron micrograph of the nonwoven cloth of BAL-1 after operating in the bioartificial liver therapy. Cells 14 attach to the fibers 15 of the nonwoven cloth satisfactorily.

FIG. 12 is a scanning electron micrograph of the hollow fiber membrane of BAL-1 after operating in the bioartificial liver therapy. Cells do not attach to the surface of the hollow fiber membrane 16 at all.

### BEST MODE FOR CARRYING OUT THE INVENTION

In the present invention, the terms “reversibly immortalized” or “capable of reversibly proliferating” means that a cell is brought into a condition capable of proliferating immortally by transducing an immortalizing gene into the cell; after proliferation of the cells up to a desired cell number, the cell division is arrested by excising the immortalizing gene so that the condition of the cell is reverted to the former condition with high safety.

Mammalian liver cells employed in the present invention are, for example, liver cells of swine, monkey, anthropoid, and human; preferably human liver cells. For example, human adult liver cells and human fatal liver cells can be employed in the present invention.

The term “liver cell” employed in the present invention means that a cell has ability to produce protein such as albumin and various blood coagulation factors which are index of liver function, ability of gluconeogenesis, ability to produce urea, abilities of detoxication and purification of blood, and ability to metabolize amino acid, glucide and lipid. Examples thereof include hepatocyte, liver sinusoid endothelial cell, liver stellate cell, Pit cell, and Kupffer cell.

A liver cell employed in the present invention is commercially available (e.g. Sanko Junyaku Co., Ltd. and Dainippon Pharmaceutical Co., Ltd.).

The term “site-specific recombination sequence” described herein means a specific sequence recognized by a site-specific recombination enzyme. When a pair of the sequences in the same direction is located in the same DNA molecule, DNA strand is excised between the sequences.

As the site-specific recombination sequence, LoxP sequence, FRT sequence, and the like are exemplified. LoxP sequence is preferable. The LoxP sequence is a known site-specific recombination sequence recognized by Cre recombinase. When a pair of LoxP sequences in the same direction is located in the same DNA molecule, Cre recombinase derived from P1 phage of Escherichia coli can a series of reactions by itself: 1) it recognizes the LoxP sequence comprising 34 bases, and 2) it binds to the site thereof, and excises the DNA sequence encoded between the LoxP sequences.

Therefore, a gene encoded between a pair of the LoxP sequences can be excised by Cre recombinase later.

The term “immortalizing gene” described herein means a gene capable of dividing infinitely, and for example, SV40T gene and human telomerase reverse transcriptase (hTERT) gene, and the like are exemplified.

The above-mentioned SV40T gene is a known tumor antigen (T antigen) gene of DNA type tumor virus.

The above-mentioned hTERT is derived from TERT gene in a normal cell. hTERT gene is a gene that enhanced expression thereof naturally in stem/progenitor cells in organs repeatedly regenerating over a lifetime such as blood, skin, intestinal mucosa, endometrium and the like, as well as in lymphocytes clonally proliferating with every exposure to a specific antigen.

The term “suicide gene” described herein means a gene encoding an enzyme derived from bacteria or virus, and having the function that it metabolically converts a prodrug with a low activity to a substance having a strong activity of damaging a cell. The cell into which such a suicide gene is transferred is killed selectively by administration of a prodrug.

Accordingly, when a suicide gene is transferred to a targeted cell, the targeted cell can be selectively removed depending upon purpose by administration of a prodrug. As representative combinations of a suicide gene and a prodrug, there are a combination of herpes simplex virus-thymidine kinase (HSVTK) gene and ganciclovir used in clinical test as an antiviral drug, a combination of cytosine deaminase gene in Escherichia coli and 5-fluorocytosine being an antibacterial agent, and the like (see Elion G B. Am. J. Med. 73, 7, 1982; Craig A M. Proc. Acad. Sci. USA 89, 33, 1992). As a suicide gene employed in the present invention, HSVTK gene is preferable in that its efficacy was already proved in animal tests (see Moolten F L, Wells J M: J Natl Cancer Inst. 82: 297-300, 1990; Culver K W, Ram Z, Wallbridge S, Ishii H, Oldfield E H. Science 1992; 256; 1550), and that its efficacy was also verified in clinical application of a gene therapy of human prostate cancer.

A reversibly immortalized mammalian liver cell employed in the present invention can be prepared by transferred the DNA sequence encoding an immortalizing gene interposed between a pair of site-specific recombination sequences and a suicide gene located in the outside of the pair of the site-specific recombination sequences to a mammalian liver cell with a vector.

As a vector, both a viral vector and non-viral vector can be used. A viral vector is preferable in that efficient gene transfer is possible by only filtrating culture supernatant of the cell to yield a recombinant vector, and adding it onto the targeted cell. However, the viral vector has problems including tumors caused by structural LTR and a defect that the number of transferred copy would be excess. Therefore, using non-viral vector is preferable.

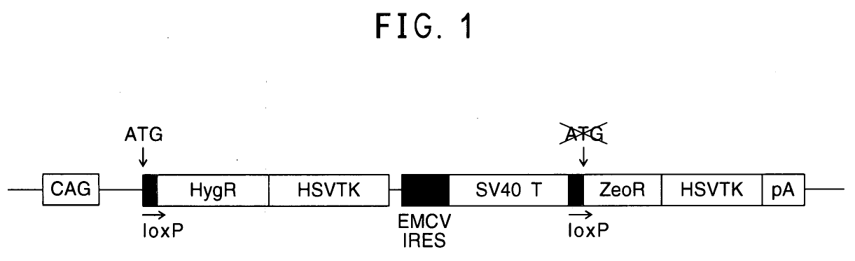

As a this kind of vector, for example, non-viral vector pYK-1 (see FIG. 1), including non-viral promoter CAG, encoding SV40T gene, hygromycin-resistant gene (HygR)/herpes simplex virus-thymidine kinase (HSVTK) fusion gene in between a pair of LoxP sequences being a target of Cre recombinase, and zeosin resistant gene (ZeoR)/HSVTK fusion gene in the outside of the LoxP sequences all together, is usable. Herein, as a non-viral promoter, CAG promoter or CMV (cytomegalovirus) promoter is usable. However, in terms of enhancement of expression, CAG promoter is preferable. Furthermore, CAG comprising cytomegalovirus IE enhancer, chicken β-actin promoter and rabbit β-globin polyadenylation signal, a person skilled in the art can produce it by reference to following the article (see Kanegae Y, Takamori K, Sato Y, et al., Gene (1996) 181: 207-212).

As the method of transferring a non-viral vector, there is electroporation using calcium phosphate transfection, lipofection method, Nucleofector™ method, and the like. In terms of transfection efficiency, electroporation using nucleofector and the like is preferable.

As the gene transfer, establishment of immortalized cell line and passage culture, cell culture in serum-free medium is preferable. As serum-free medium, there is CS-C serum-free medium or ISE-RPMI or HGM (hepatocyte growth medium). In terms of availability, for example, CS-C serum-free medium available from Cell System Co., Ltd. (Seattle, Wash., USA) or Dainippon Pharmaceutical Co., Ltd. is preferable.

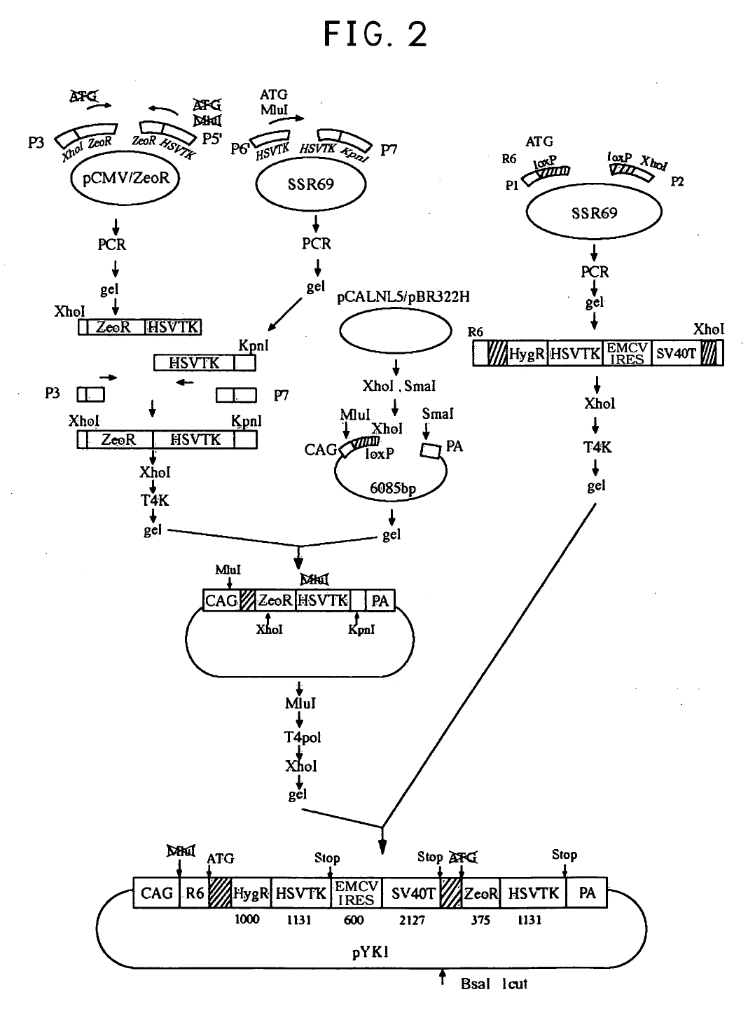

For the reasons stated above, among reversibly immortalized mammalian liver cells, CYNK-1 cell line (deposited in International Patent Organism Depository of National Institute of Advanced Industrial Science and Technology, address: AIST Tsukuba Central 6, 1-1, Higashi 1-Chome Tsukuba-shi, Ibaraki-ken 305-8566 Japan, deposited date: Mar. 10, 2004, accession number: FERM BP-08657) established by transferring non-viral vector pYK-1 to normal human hepatocyte and a passage cell line thereof are the most preferable. Furthermore, the reversibly immortalized mammalian liver cell in the present invention has so-called morphologic features of parenchymal cells of the liver that have an abundance of intracellular granules 3 having a large nucleus 2 with some nucleoli 1 as shown in FIG. 2 (CYNK-1 cell (deposited in International Patent Organism Depository of National Institute of Advanced Industrial Science and Technology, address: AIST Tsukuba Central 6, 1-1, Higashi 1-Chome Tsukuba-shi, Ibaraki-ken 305-8566 Japan, deposited date: Mar. 10, 2004, accession number: FERM BP-08657)).

The reversibly immortalized mammalian liver cell in the present invention can remove an immortalizing gene by affecting a site-specific recombination enzyme, as mentioned above. The recombination enzyme can be selected for a site-specific recombination sequence by a person skilled in the art, as mentioned above.

As a method of affecting a site-specific recombination enzyme, for example, in Cre recombinase case, there are (1) a method of transduction of Cre recombinase by adenoviral vector capable of efficient gene transfer, (2) a method of adding Cre recombinase in cell culture solution in calcium phosphate transfection, (3) a method of adding TAT protein derived from human immumodeficiency virus (HIV) and Cre recombinase in culture supernatant, (4) a method of adding cationic Cre recombinase in cell culture solution, and (5) a method of adding a system capable of deleting the gene freely with addition of an agent, by transferred a drug-induced Cre recombinase enzyme expression vector.

A reverted cell, by deletion of immortalizing gene from the reversibly immortalized mammalian liver cell in the present invention or the passage cell line thereof, expands slightly compared to a cell before reversion and maintains so-called morphologic features of parenchymal cells of the liver that have an abundance of intracellular granules having a nucleus with some nucleoli.

Meanwhile, as a liver cell to improve liver malfunction, some biochemical requirements must be met. To be concrete, i) alleviation of bilirubin plasma or jaundice, ii) improvement of hepatic encephalopathy caused by hyperammonemia, iii) detoxification and clarification of blood, iv) resupply of albumin or blood coagulation factor and the like. Specially, alleviation of hyperammonemia is important for prevention of hepatic encephalopathy and brain-dead development (see Strom S C. Transplantation 63, 559, 1997). Glutamine synthetase (GS) is a main enzyme in removing ammonia, and the expression thereof is important to a hepatic cell line established for improvement of liver malfunction (see Strom S C. Transplantation 63, 559, 1997).

Therefore, it is preferable that the reversibly immortalized mammalian liver cell in the present invention and the passage cell thereof as well as the reverted cell thereof expresses at least any of albumin gene, bilirubin-UGT gene, GS gene, GST-π gene and HBCF-X gene. These all genes being expressed are more preferable.

This kind of the reversibly immortalized mammalian liver cell in the present invention and the passage cell thereof as well as the reverted cell thereof are applicable to therapy of various kinds of liver diseases as a bioartificial liver or a cell preparation.

As “a liver disease” in the present invention, there include liver failure such as acute liver failure depending on virus, agent and intoxication (for example, toadstool and the like); liver-based metabolic diseases such as hemophilia, α1-antitrypsin deficiency, galactosemia, hepatorenal tyrosinemia, maple syrup urine disease, glycogenosis type 1a, hepatic porphyria, hypobetalipoproteinemia, hypercholesterolemia, primary hyperoxaluria type 1, Crigler-Najjar syndrome type 1, hyperphenylalaninemia; acute on chronic liver disease and the like. It is preferable that the medical agent of the present invention uses for treatments of liver failure and liver-based metabolic diseases.

As a cell preparation in the present invention, there are suspension that suspend the reversibly immortalized mammalian liver cell in the present invention and the passage cell thereof as well as the reverted cell thereof in medium, isotonic solution, or buffer solution, or clumps of cells including pellet condensed by centrifugation and the like. Medium, isotonic solution, or buffer solution can be properly selected to match with the liver cell. Moreover, the cell preparation can be frozen by adding cryoprotective agent including DMSO. And as the cell preparation, in view of inoculation into human bodies, the reverted cell after deleting an immortalizing gene is preferable. To use safely, a cell preparation can be treated under the conditions being the level of protein denaturation of disease-causing cells with maintaining a function of a cell preparation, such as treatment with heat, radiation, or mitomycin C and the like.

As an administration form (transplantation method) of the cell preparation, intraportal injection, or intraperitoneal injection, intrasplenic arterial injection are available. Among them, the intraportal injection and the intrasplenic arterial injection are more preferable, the intraportal injection is the most preferable. As the dosage (amount of transplantation) of the therapeutical cell preparation, 1×10⁸ to 1×10¹⁰ cells are preferable, more preferably 5×10⁸ to 1×10¹⁰ cells, 1×10⁹ to 1×10¹⁰ cells are the most preferable. And the dosage (amount of transplantation) can be properly altered based on age, weight, symptom of a patient.

As the bioartificial liver, for example, there is a hybrid type artificial liver wherein a hollow fiber type reactor (device) and separated/cultured cells are combined. There are three types of the bioartificial liver: the first is fixed on the outside of the body and connected with blood vessel; the second is fixed in the body and connected with blood vessel; and the third is fixed in the abdominal cavity and not connected with blood vessel. Both the reversibly immortalized mammalian liver cell and the reverted cell thereof in accordance with the present invention are applicable to any type of bioartificial liver.

In the development of bioartificial liver, design/development of a reactor are also important factors. As the bio-reactor, various types are known including HepatoAssist for a bioartificial liver therapy using swine hepatocytes (see Hui T, Rozga J, Demetriou A A. J Hepatobiliary Pancreat Surg 2001; 8: 1-15.) developed by Demetriou et al. in Cedars-Sinai Medical Center (Los Angeles, Calif., USA) with the support of Circe Biomedical Inc. (Lexington, Mass., USA), and MELS (Modular Extracorporeal Liver System) using swine hepatocytes developed by Gerlach (German). These reactors are also applicable to the present invention. However, cells are filled simply in the space inside or outside the hollow fibers and are inclined to suspend because there is no scaffold for adhesion of hepatocyte. Hepatocytes tend not to differentiate sufficiently in a suspending state, and tend to receive stimulation of stress due to collisions with other cells.

Therefore, in the present invention, a reactor comprising hollow fibers and a nonwoven cloth to provide a scaffold for hepatocyte is preferable.

As the hollow fiber membrane, any kind of hollow fiber is usable, unless cells adhere to the surface of the membrane and prevent matter exchange. Specifically, those conventionally marketed for medical use, for example, polysulfone membrane, saponified ethylene-vinyl acetate random copolymer membrane (e.g. trade name: EVAL, manufactured by Kuraray Co., Ltd.) and the like are preferable. The hollow fiber membranes on the market include dialysis membrane (pore size: up to 5 nm), plasma constituent separation membrane (pore size: 20 to 30 nm), and plasma separation membrane (pore size: 30 to 200 nm), depending upon the pore size. In view of transmission of substances, plasma separation membrane (pore size: 30 to 200 nm) is preferable.

As the nonwoven cloth, nonwoven cloth processed/modified so that cells adhere thereto is preferable. Among them, a polytetrafluoroethylene (PTFE) nonwoven cloth treated with polyamino acid urethane is preferable in terms of easy processing.

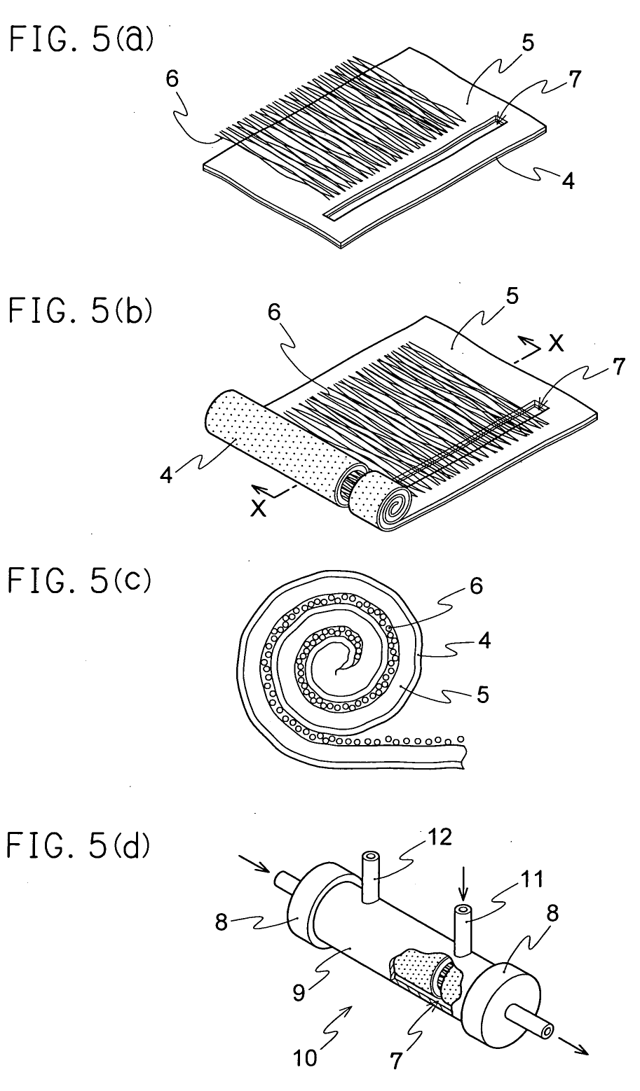

FIG. 5 shows a procedure up to housing with respect to an embodiment of a bioartificial liver reactor in accordance with the present invention. Hollow fibers 6 are placed on a nonwoven cloth 5 provided with a backing material 4 (FIG. 5(a)). The nonwoven cloth 5 provided with the backing material 4 is provided with a slit 7. The resultant is wound into a roll (FIG. 5(b)). FIG. 5(c) is a cross section of FIG. 5(b) taken on line X-X. The roll is incorporated into a cylindrical container 9 with members 8 for preventing liquid leak provided at its both ends. The reactor is provided with an inlet 11 for cells and an outlet 12 capable of sampling cells, and the slit 7 is arranged so as to communicate with the cell inlet 11.

Besides, bioartificial liver therapy is preferably conducted by use of an apparatus with the integration of the following functions: 1) monitoring inflow pressure and outflow pressure of bioartificial reactor in real time; 2) alarming when air bubbles are formed; 3) warming of the reactor (37° C.), and the like.

The present invention is more specifically explained by means of Examples. However, it is to be understood that the invention is not limited to only these Examples.

### EXAMPLE 1

### Preparation of Non-Viral Vector pYK-1

A non-viral vector pYK-1 (see FIG. 1) was prepared by the following procedure (see FIG. 2).

(1) To obtain a fragment of zeosin resistant gene (ZeoR), pCMV/ZeoR (available from Invitrogen) was used as a template. A primer P3 integrating XhoI at the 5′ side and a primer P5′ integrating HSVTK at the 3′ side were designed at both sides of the zeosin region of the pCMV/ZeoR. Using these primers and the template, PCR reaction was performed under the conditions: at 95° C. for 5 minutes, (at 95° C. for 30 seconds, at 60° C. for 30 seconds, and at 72° C. for 2 minutes)×30 cycles and then at 72° C. for 15 minutes. Zeosin initiation codon of the primer P3 was deleted in order not to express the zeosin gene.

(2) To obtain a HSVTK fragment, SSR#69 (see Westerman K A, Leboulch P., Proc. Natl. Acad. Sci. USA 93: 8971-8976, 1996) was used as a template. A primer P6′ and a primer P7 were designed at both sides of the HSVTK region of the SSR#69. Using these primers and the template, PCR reaction was performed under the conditions: at 95° C. for 5 minutes, (at 95° C. for 30 seconds, at 60° C. for 30 seconds, and at 72° C. for 2 minutes)×30 cycles and then at 72° C. for 15 minutes.

(3) To obtain zeosin/HSVTK connecting fragment, a mixture of the zeosin fragment collected in (1) above and the HSVTK fragment collected in (2) above was used as a template. PCR reaction using primers P3 and P7 as well as the templates was performed under the conditions: at 95° C. for 5 minutes, (at 95° C. for 30 seconds, at 61° C. for 1 minute, at 72° C. for 3 minutes)×30 cycles and then at 72° C. for 15 minutes. By the PCR reaction, zeosin and HSVTK fragments were connected, because the 3′ side of the primer P5′ and the 5′ side of the primer P6′ were complementarily designed.

(4) To obtain a fragment containing hygromycin-resistant gene, HSVTK gene, EMCV-IRES gene and SV40T gene, SSR#69 was used as a template. Primers P1 and P2 were designed at both sides of the fragment of the SSR#69. PCR reaction using the primers and the template was performed under the conditions: at 95° C. for 5 minutes, (at 95° C. for 30 seconds, at 60° C. for 1 minute, at 72° C. for 5 minutes)×30 cycles and then at 72° C. for 15 minutes.

HCV5′UTR and LoxP were integrated at the 5′ side of the primer P1, and LoxP and XhoI were integrated at the 3′ side of the primer P2.

The PCR products obtained were electrophoresed using a gel containing 1.2% of a mixture of NuSieve agarose and SeaKem agarose at a ratio of 3:1), and then purified by using DNACELL (available from Daiichi Pure Chemicals Co., Ltd.) to be collected.

Secondly, the zeosin/HSVTK connecting fragment was treated with XhoI and T4PNK, which was inserted into XhoI/SmaI-treated LoxP switching expression vector pCALNL5/pBR322H using LigaFast Rapid DNA Ligation System (available from Promega). The pCALNL5/pBR322H was constructed by replacing pUC Ori in pCALNL5 (available from Riken Bank RDB-1862) by pBR322 Ori. The zeosin/HSVTK inserted expression vector pCALNL5/pBR322H was transformed into competent cells (JM109) by electroporation, which were selected by LB plate with ampicillin. Clones were treated with Mlu I, T4pol, and Xho I, in which the Xho I/T4K-treated fragment containing hygromycin-resistant gene, HSVTK gene, EMCV-IRES gene and SV40T gene, was inserted. The fragment-inserted clone was transformed into competent cells (JM109), which were selected by LB plate with ampicillin. In this way, pYK1 was constructed.

### EXAMPLE 2

### Establishment of Reversibly Immortalized Human Hepatic Cell Line CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken 305-8566 Japan, Deposited Date: Mar. 10, 2004, Accession Number: FERM BP-08657)

Normal human hepatocytes (passage number: 1) (CS-ABI-3716, available from Dainippon Pharmaceutical Co., Ltd.) with 80% confluent was treated with trypsin in a T-25 flask to obtain human hepatocytes (1×10⁶ cells). The cells were diluted with 1 ml of buffer for Nucleofector™ system (Nucleofector™ solution, available from Wako Pure Chemical Industries, Ltd.), followed by adding 2 μl of a suspension containing pYK-1 plasmid DNA (1 μg/μl) in TE buffer (available from Sigma). Gene transfer of pYK-1 plasmid DNA was performed by Nucleofector™ system (Nucleofector™ system, available from Wako Pure Chemical Industries, Ltd.) according to the system's protocol. The obtained cells were inoculated in a T-25 flask, followed by culturing using CS-C serum free medium (CS-SF-4Z0-500, available from Dainippon Pharmaceutical Co., Ltd.). After 48 hours from the gene transfer, resistant clones were selected by CS-C serum free medium with 100 μg/ml hygromycin. After two weeks from the starting of selection, the resistant clones were observed. After four weeks from the selection starting, CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken 305-8566 Japan, deposited date: Mar. 10, 2004, accession number: FERM BP-08657) was established by using cloning ring.

The cell was deposited at International Patent Organism Depository, National Institute of Advanced Industrial Science and Technology (address: AIST Tsukuba Central 6, 1-1, Higashi 1-Chome, Tsukuba-shi, Ibaraki-ken 305-8566 Japan, deposited date: Mar. 10, 2004, accession number: FERM BP-08657).

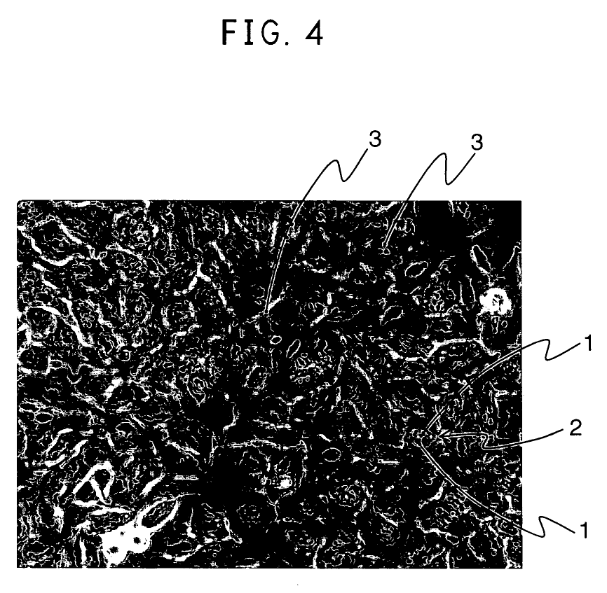

The obtained CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) showed characteristics of parenchymal cells of the liver; the cell was abundant in intracellular granules 3 and had a large nucleus 2 with several nucleoli 1 (see FIG. 4). The CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) was immortalized without a crisis in growth inhibition, proliferated as a single layer in the CS-C serum free medium and then the number thereof was doubled in about 48 hours.

### EXAMPLE 3

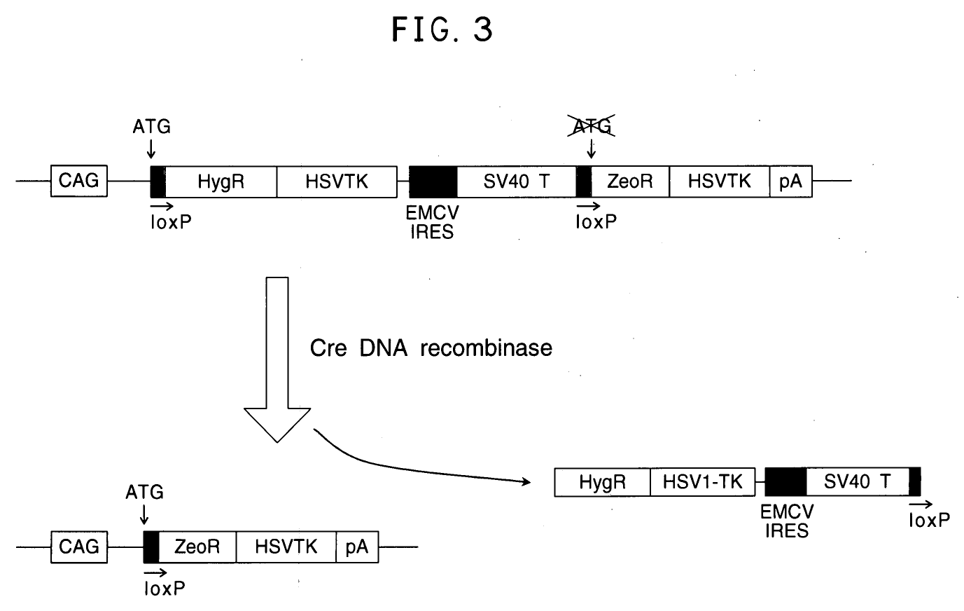

### Excision of SV40T Gene Using Cre Recombinase (see FIG. 3)

The CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) was infected with replication-incompetent recombinant adenoviral vector AxCANCre (3×10⁸ pfu/ml) (Available from Riken Gene Bank, Japan, RDB No. 1748) producing Cre recombinase labeled by nuclear localization signal (NLS), at 5 MOI (multiplicity of infection). After the infection with AxCANCre for 1 hour, the cells were collected by treating with trypsin and selected with zeosin (culturing in the CS-C serum free medium containing 5 μg/ml of zeosin (available from Sigma)) for 5 days, which achieved the complete exclusion of SV40T gene expression cells. By transient expression of Cre recombinase, excision of DNA between LoxP sequences was performed.

The obtained cells grew somewhat larger than the cells before reversion and maintained morphological features of parenchymal cells of the liver; that is, the cells had a large nucleus with several nucleoli and were abundant in intracellular granules.

### TEST EXAMPLE 1

### Gene Expression in CYNK-1 Cell (Deposited with International Patent Organism Depository, National Institute of Advanced Industrial Science and Technology, Address: AIST Tsukuba Central 6, 1-1, Higashi 1-Chome, Tsukuba-shi, Ibaraki-ken, 305-8566 Japan, Deposited Date: Mar. 10, 2004, Accession Number: FERM BP-08657) Before and After Excision of SV40T Gene

With respect to the CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) before and after excision of SV40T gene, the expression of important genes related to metabolism in liver, i.e. albumin gene, albumin gene, ASGPR gene, bilirubin-UGT gene, GS gene, GST-π gene and HBCF-X gene, and SV40T gene were assayed by RT-PCR method. In the RT-PCR method, RNA was extracted from the CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) using RNAzol (available from Cinna/BioTecx, Friendswood, Tex., USA) and 1 μg of the resulting total RNA was reverse-transcribed with RNA reverse transcriptase at 22° C. for 10 minutes and then at 42° C. for 20 minutes.

The 2 μg of reverse-transcribed products obtained underwent PCR amplification using 20 pmol/ml of each primer and AmpliTag Gold Kit (available from PerkinElmer Cetus, Norwalk, Conn., USA) according to the protocol. PCR reaction was performed as follows: incubation at 95° C. for 10 minutes, 35 cycles of incubation consisting of incubation at 95° C. for 30 seconds, at 60° C. for 30 seconds and at 72° C. for 30 seconds, and final incubation at 72° C. for 7 minutes. As primers for each gene, the following primers were used.

| | |
|---|---|
| albumin gene (576 bp) | |
| 5′ primer: AAACCTCTTGTGGAAGAGCC | (SEQ No. 1) |
| 3′ primer: CAAAGCAGGTCTCCTTATCG | (SEQ No. 2) |
| ASGPR gene (495 bp) | |
| 5′ primer: TAGGAGCCAAGCTGGAGAAA | (SEQ No. 3) |
| 3′ primer: ACCTGCAGGCAGAAGTCATC | (SEQ No. 4) |
| bilirubin-UGT gene (495 bp) | |
| 5′ primer: ATGACCCGTGCCTTTATCAC | (SEQ No. 5) |
| 3′ primer: TCTTGGATTTGTGGGCTTTC | (SEQ No. 6) |
| GST-π gene (496 bp) | |
| 5′ primer: GCCCTACACCGTGGTCTATT | (SEQ No. 7) |
| 3′ primer: GGCTAGGACCTCATGGATCA | (SEQ No. 8) |
| GS gene (535 bp) | |
| 5′ primer: ATGCTGGAGTCAAGATTGCG | (SEQ No. 9) |
| 3′ primer: TCATTGAGAAGACACGTGCG | (SEQ No. 10) |
| HBCF-X gene (493 bp) | |
| 5′ primer: GTGCATGGAAGAGACCTGCT | (SEQ No. 11) |
| 3′ primer: GAAGTCAAGCAGGTCGAAGG | (SEQ No. 12) |
| SV40T gene (422 bp) | |
| 5′ primer: CAGGCATAGAGTGTCTGC | (SEQ No. 13) |
| 3′ primer: CAACAGCCTGTTGGCATATG | (SEQ No. 14) |

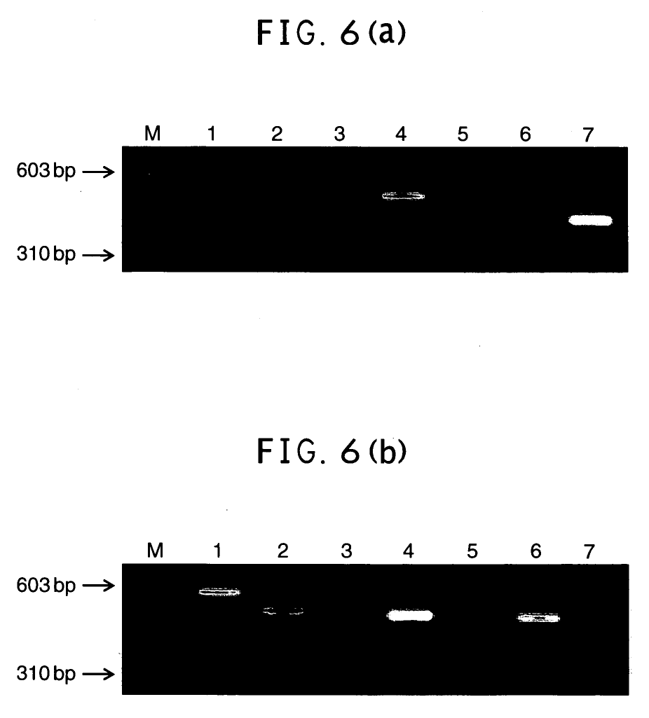

Before and after the reversible immortalization of cells, expressions of these hepatic function genes were maintained and excision of SV40T gene by Cre/LoxP recombination was observed (see FIGS. 6(a) and 6(b)). These results indicate that it is possible to manufacture physiological active substances such as albumin and blood coagulation factors by use of the CYNK-1 cell (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657), which are applicable to the development of cellular pharmaceutical products.

### TEST EXAMPLE 2

### Ganciclovir Sensitivity of CYNK-1 Cell (Deposited with International Patent Organism Depository, National Institute of Advanced Industrial Science and Technology, Address: AIST Tsukuba Central 6, 1-1, Higashi 1-Chome, Tsukuba-shi, Ibaraki-ken, 305-8566 Japan, Deposited Date: Mar. 10, 2004, Accession Number: FERM BP-08657) Before and After Excision of SV40T Gene

Two hundred CYNK-1 cells (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) were inoculated in a 96-well plate, cultured in CS-C serum free culture medium alone or CS-C serum free culture media containing various concentrations of ganciclovir (5, 10, 20, 50 or 100 μM) and then cell proliferation was investigated by MTT assay (see Mcsmann T. J., Immunol. Methods, 65, 55, 1983).

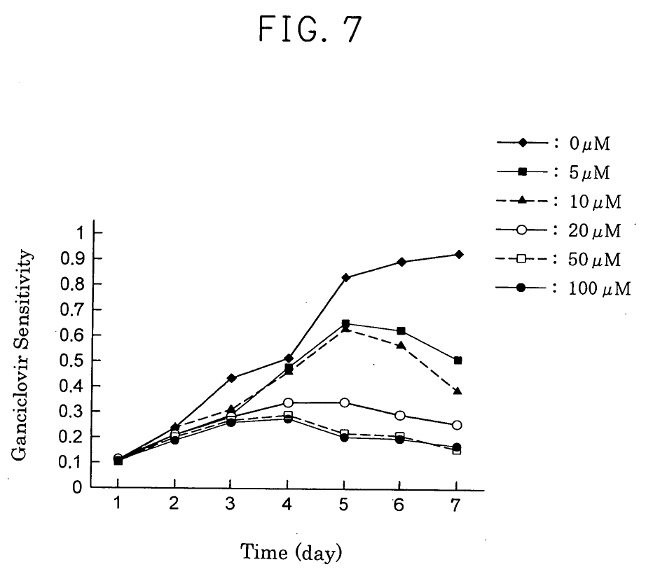

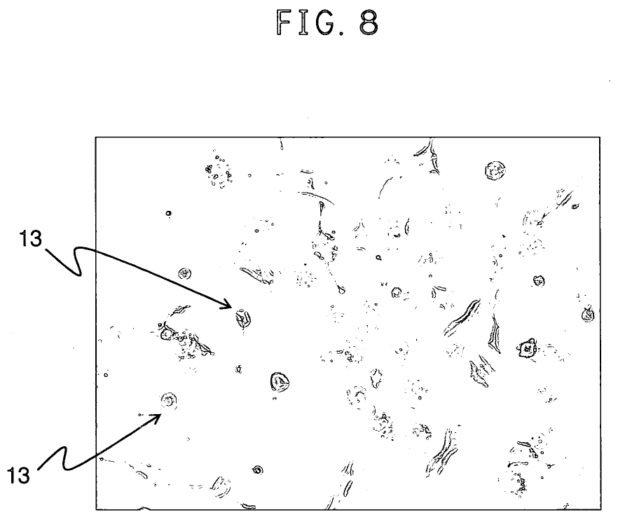

Cell viability is shown in FIG. 7 as relative sensitivity. Ganciclovir sensitivity of the CYNK-1 cell (deposited with International Patent Organism Depository, National Institute of Advanced Industrial Science and Technology, address: AIST Tsukuba Central 6, 1-1, Higashi 1-Chome Tsukuba-shi, Ibaraki-ken, 305-8566 Japan, deposited date: Mar. 10, 2004, accession number: FERM BP-08657) depends on the concentration of ganciclovir. One-week cultivation with 5 μM of GCV stopped cell proliferation and some of the cells died (see FIGS. 7 and 8).

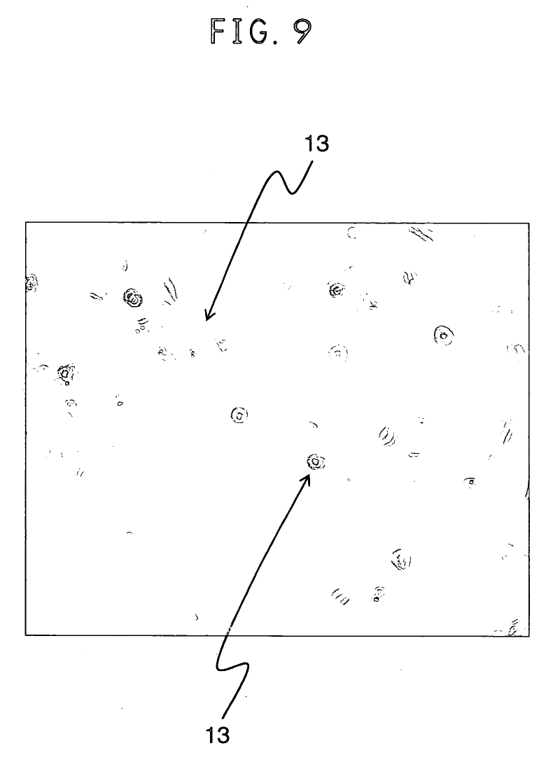

The reverted CYNK-1 cells also showed sensitivity to GCV. One-week cultivation in the presence of 5 μM of GCV stopped cell proliferation and some of the cells died (see FIG. 9). All of the cells died within 10 days.

These data suggest that cell proliferation could be controlled by administration of ganciclovir.

### TEST EXAMPLE 3

### Treatment Effect of Reverted CYNK-1 Cell on Liver Failure

With use of a cell culture flask T-225 (available from Corning, N.Y., USA), the CYNK-1 cell (deposited with International Patent Organism Depository, National Institute of Advanced Industrial Science and Technology, address: AIST Tsukuba Central 6, 1-1, Higashi 1-Chome Tsukuba-shi, Ibaraki-ken, 305-8566 Japan, deposited date: Mar. 10, 2004, accession number: FERM BP-08657) was incubated in a culture medium A (CS-C serum free medium (CS-SF-4Z0-500, available from Dainippon Pharmaceutical Co., Ltd.) containing 0.1 mg/l of streptomycin and 100 U/ml of penicillin G) in an incubator wherein the room temperature was 37° C. and CO₂ concentration was 5%. The CYNK-1 cell (deposited with International Patent Organism Depository, National Institute of Advanced Industrial Science and Technology, address: AIST Tsukuba Central 6, 1-1, Higashi 1-Chome Tsukuba-shi, Ibaraki-ken, 305-8566 Japan, deposited date: Mar. 10, 2004, accession number: FERM BP-08657) proliferated, so that 2×10⁷ cells were obtained per flask. The cells were detached from the surface of the flask by using 8 ml of 0.25% trypsin solution containing 0.02% EDTA in PBS (phosphate buffered physiological saline), and 40 ml of the culture medium A was added thereto, and the resultant was stirred and centrifuged under the conditions of 4° C., 800 rpm, and 7 minutes to collect the cells. 30 ml of the culture medium A was added to the obtained CYNK-1 cells (deposited with International Patent Organism Depository, National Institute of Advanced Industrial Science and Technology, address: AIST Tsukuba Central 6, 1-1, Higashi 1-Chome Tsukuba-shi, Ibaraki-ken, 305-8566 Japan, deposited date: Mar. 10, 2004, accession number: FERM BP-08657) (6×10⁷ cells in total for three cell culture flasks T-225), and the resultant was stirred. Then, the cells were seeded into a roller bottle (formal name: 1700 cm² Expanded Surface Roller Bottle, available from Corning) and 500 ml of the culture medium A was added thereto and the resultant was cultured. The culture in a large scale was performed by utilizing a cell preparation roller apparatus (available from Bellco) using four roller bottles. When the cells became confluent condition, recombination was explored by adding the adenoviral vector AxCANCre. 48 hours after addition of the AxCANCre, the cells were cultivated for five days in zeosin containing culture medium, and the cells were collected in the same trypsin treatment as above. 1.2×10⁹ reverted cells were obtained using four roller bottles (3×10⁸ cells/roller bottle).

10 female swines (5 kg) of Landrace White purchased at agricultural cooperative in Bisei-cho, Okayama-ken were given nothing to eat for 12 hours before operation. 0.5 mg of Atropine sulfate (available from Fuso Pharmaceutical Industries, Ltd. Osaka, Japan) and 3 mg of Ketaral for animal (Ketamine hydrochloride) (available from Sankyo Co., Ltd., Tokyo, Japan) were injected into muscle, and after remission, 1 mg of Droleptan (Droperidol) (available from Sankyo Co., Ltd., Tokyo, Japan), and 2 mg of Myoblock (Pancuronium bromide) (available from Organon International Inc., Netherlands) were injected intravenously, and endotracheal intubation was conducted. While anesthesia was maintained by means of a ventilator using oxygen, laughing gas, and Sevoflurane, left external cervical vein was exposed, and cannulation of 6Fr Atom tube (available from Atom Medical Corporation, Tokyo, Japan) was conducted. After laparotomy through an abdominal midline incision, cannulation of 6Fr Atom tube (available from Atom Medical Corporation, Tokyo, Japan) was conducted through splenic vein so that the tip thereof was put in the parent trunk of the portal vein. It was confirmed by palpation that the tip was located in the parent trunk of the portal vein. Each end of Atom tube was secured with sutures on the surface of the skin after formation of subcutaneous tunnel. Before and after the operation was performed, 0.5 g of Sepatren (cephalosporin antibiotic) (available from Sumitomo Pharmaceuticals Co., Ltd., Osaka, Japan) was injected intravenously. After the operation, the endotracheal tube was extubated after confirming the complete awakening. Immediately thereafter, 0.5 g/kg of D-galactosamine (available from Sigma) dissolved in 50 ml of physiological saline was injected intravenously through Atom tube inserted in left external cervical vein over 10 minutes, thereby inducing drug-induced hepatitis.

Each Atom tube underwent a locking treatment using 1 ml of heparin (1,000 Units) to prevent formation of blood clot. 18 hours after administering D-galactosamine, 1×10⁹ reverted cells prepared by Roller Bottle culturing were diluted with 100 ml of Ringer's solution, and were transplanted over about 30 minutes (transplantation group, n=5) via portal vein through 6Fr Atom tube inserted in splenic vein. As a control experiment, the cells were not transplanted to the rest 5 female swines (control group, n=5).

To grasp whole body condition of the swines, electrocardiogram, cardiac rate, and arterial blood pressure were monitored. In the transplantation group, temporary elevation of portal pressure (25 cmH₂O) was recognized during the transplantation. However, the swines could stand the hepatic cell transplantation.

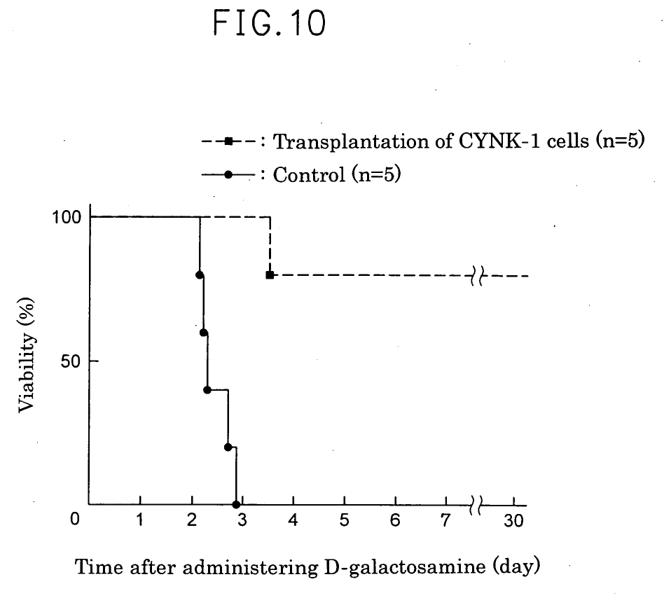

The results are shown in FIG. 10. In the control group, all swines were died from hepatic failure. However, in the reverted CYNK-1 cell transplanted group, 4 of 5 swines could survive for more than one month. Thus statistically significant difference was recognized. The data suggest that the reverted CYNK-1 cell transplantation is also effective for human hepatic failure.

### EXAMPLE 4

### Preparation of Bioartificial Liver

A bioartificial liver (BAL) (see FIG. 5) of whole blood circulating system comprising hollow fibers and a nonwoven cloth was manufactured by the procedure below. The bioartificial livers manufactured are named BAL-1 to BAL-6 according to the kind of hollow fiber membrane, as shown in Table 1.

TABLE 1

| | Hollow fiber membrane | |
|---|---|---|
| Bioartificial liver | Material | Pore size |
| BAL-1 | Polysulfone *¹ | Dialysis membrane |
| BAL-2 | Polysulfone | Plasma separation membrane |
| BAL-3 | Polysulfone | Plasma constituent separation |
| | | membrane |
| BAL-4 | EVAL *² | Dialysis membrane |
| BAL-5 | EVAL | Plasma separation membrane |
| BAL-6 | EVAL | Plasma constituent separation |
| | | membrane |

*¹ available from Kuraray Medical Co., Ltd.
*² available from Kuraray Medical Co., Ltd.

550 hollow fibers of 10 cm were regularly placed on a nonwoven cloth (10×10 cm) backed with rayon (see FIG. 5(a)), and the resulting sheet was wound into a roll (see FIG. 5(b)). The schematic sectional view of the roll is shown in FIG. 5(c). The role comprising the nonwoven cloth and hollow fibers was incorporated into a cylindrical container with members for preventing liquid leak provided at its both ends (see FIG. 5(d)). About 1×10⁹ reversibly immortalized hepatocyte CYNK-1 (deposited with International Patent Organism Depository (IPOD), National Institute of Advanced Industrial Science and Technology (AIST), address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657) was suspended in 10 ml of CS-C serum free medium (CS-SF-4Z0-500, available from Dainippon Pharmaceutical Co., Ltd.), and the resulting suspension was filled in the reactor manufactured.

As the nonwoven cloth, PTFE (polytetrafluoroethylene) nonwoven cloth (Kuraray Medical Co., Ltd.) treated with polyamino acid urethane (PAU) having adhesiveness to cell was used.

To evaluate biocompatibility of the obtained reactors, each reactor was provided between carotid artery and cervical vein of healthy swine and operated. The reactor was able to be operated without adverse affect on 24-hour circulatory dynamics. Besides, decrease of blood cell constituents including erythrocyte and platelet was not recognized.

Furthermore, in order to conduct BAL therapy safely and scientifically, an apparatus with the integration of the following functions: 1) monitoring inflow pressure and outflow pressure of bioartificial reactor in real time; 2) alarming when air bubbles are formed; and 3) warming of the reactor (37° C.), was developed, and favorable operation of the system were verified by experiment using swine.

### TEST EXAMPLE 4

### Therapeutic Effect of Bioartificial Liver on Liver Failure

The safety and effectiveness of the bioartificial liver prepared in Example 4 were examined using cynomolgus monkeys passed quarantine inspection (available from CLEA Japan, Inc.) according to handling guideline of institutional animals.

A male cynomolgus monkey (4 kg, n=6) was intravenously injected with 0.5 g/kg of D-galactosamine (available from Sigma) to induce a drug-induced hepatitis.

18 hours after administering D-galactosamine, Atom 6Fr catheters (available from Atom Medical Corporation, Tokyo, Japan) were inserted in left internal carotid artery and in left external cervical vein of the monkey of treatment group under general anesthesia (n=2), and reactor BAL-1 prepared in Example 4 was connected therebetween. Using PERISTA BIO-MINIPUNP (available from ATTO, Tokyo, Japan) as a pump, extracorporeal circulation was performed for 6 hours at a flow rate of 10 ml/min. As an intravenous anesthesia, duplivan was used (5 ml/hour). As an antithrombogenic treatment, heparin 1,000 Units were injected in the inside of the reactor just before blood passed the reactor, and during extracorporeal circulation whole-body heparinization was performed by continuous heparin injection (500 Units/hour).

By using a reactor filled with approximately 1×10⁹ fresh swine hepatocytes (viability>90%) instead of CYNK-1 cells (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657), BAL treatment as a positive control group (n=2) was performed for 6 hours in the same way as in the treatment group. Fresh swine hepatocytes were separated from outer region of liver, which had been removed surgically, by four-step circulation method with dispase and collagenase (see Masanobu Maruyama, Naoya Kobayashi, Toshinori Totsugawa et al. Organ Biology, Vol. 9, No. 3. 295-301, 2002).

As a negative control group (n=2), the same test was performed using an empty reactor in which cells were not filled.

As a result, in the negative control group, the two monkeys were died of liver failure within 1 week from administering D-galactosamine. At autopsy, hepatic bleed necrosis, hepatic notable atrophia, jaundiced breast/accumulated ascitic fluid were recognized. Histogenetically, a wide range of hepatic necrosis was also recognized. Meanwhile, in the positive control group using approximately 1×10⁹ swine liver cells, any monkey survived for more than 3 weeks, and there is no anomaly in the autopsy at all.

In the treatment group, the two monkeys survived for more than 3 weeks. The liver thereof was completely normalized macroscopically and histogenetically. Moreover, in the treatment group, improvement of Fisher's ratio (branched-chain amino acid/aromatic amino acid ratio) was recognized.

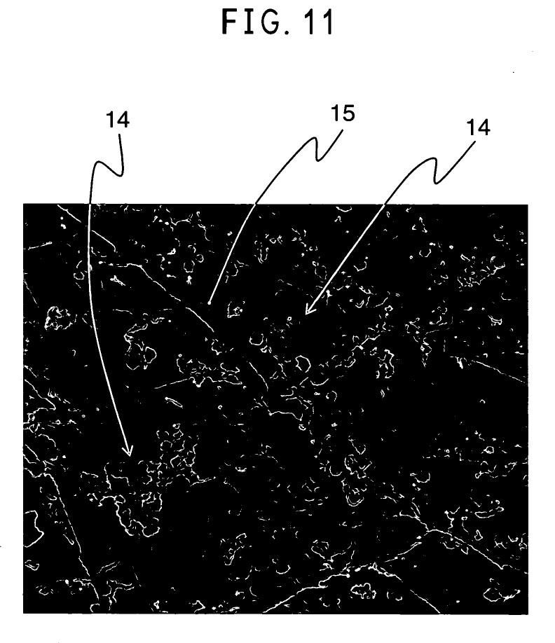

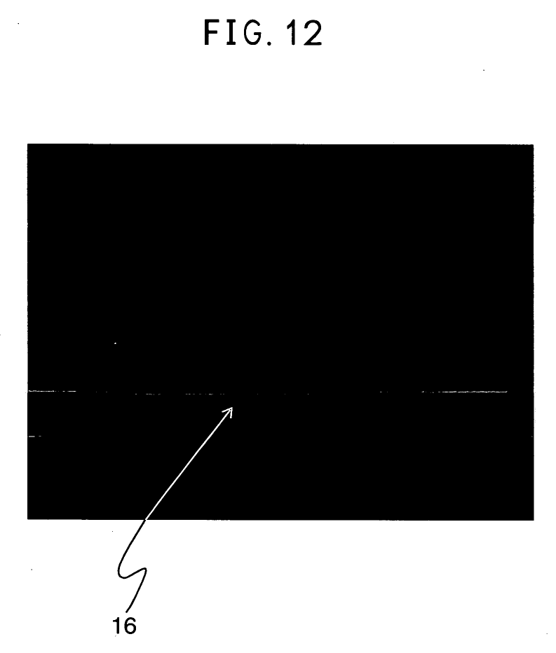

After the treatment, the reactor was fixed and adhesion of the cells was observed by scanning electron microscope. The cells satisfactorily adhered to the fibers of the nonwoven cloth (see FIG. 11), and an aggregated cluster consisting of several cells were observed. This aggregated cluster is preferable for exhibiting cellular differentiation function. Meanwhile, cells attached to the surface of the hollow fibers were not observed (see FIG. 12). This is remarkably preferable from the viewpoint of substance exchange using hollow fibers.

### INDUSTRIAL APPLICABILITY

A reversibly immortalized mammalian liver cell of the present invention enables the obtainment of the number of the liver cells, excels in safety, does not give patients cause for anxiety, enables utilization as materials for bioartificial livers and cell preparations. Furthermore, the reversibly immortalized mammalian liver cell of the present invention is applicable to development of cellulous pharmaceutical products, because it is able to produce physiologically active substances including albumin, or blood coagulation factor and the like. Besides, the reverted cell that excels further in safety and maintains the ability of a liver cell, is very useful in therapy of liver diseases.

### SEQUENCE LISTING FREE TEXT

SEQ No. 1: 5′ primer for polymerase chain reaction to detect Albumin gene
SEQ No. 2: 3′ primer for polymerase chain reaction to detect Albumin gene
SEQ No. 3: 5′ primer for polymerase chain reaction to detect ASGPR gene
SEQ No. 4: 3′ primer for polymerase chain reaction to detect ASGPR gene
SEQ No. 5: 5′ primer for polymerase chain reaction to detect Bilirubin-UGT gene
SEQ No. 6: 3′ primer for polymerase chain reaction to detect Bilirubin-UGT gene
SEQ No. 7: 5′ primer for polymerase chain reaction to detect GST-π gene
SEQ No. 8: 3′ primer for polymerase chain reaction to detect GST-N gene
SEQ No. 9: 5′ primer for polymerase chain reaction to detect GS gene
SEQ No. 10: 3′ primer for polymerase chain reaction to detect GS gene
SEQ No. 11: 5′ primer for polymerase chain reaction to detect HBCF-X gene
SEQ No. 12: 3′ primer for polymerase chain reaction to detect HBCF-X gene
SEQ No. 13: 5′ primer for polymerase chain reaction to detect SV40T gene
SEQ No. 14: 3′ primer for polymerase chain reaction to detect SV40T gene

1. A reversibly immortalized mammalian liver cell line or a passage cell line thereof, containing an immortalizing gene interposed between a pair of site-specific recombination sequences and a suicide gene in the outside of the pair of site-specific recombination sequences, wherein the suicide gene can exhibit its function after excision of the pair of site-specific recombination sequences.

2. The reversibly immortalized mammalian liver cell line or the passage cell line thereof of claim 1, wherein said mammalian is human.

3. The reversibly immortalized mammalian liver cell line or the passage cell line thereof of claim 1, wherein a promoter derived from virus is not contained.

4. The reversibly immortalized mammalian liver cell line or the passage cell line thereof of claim 1, wherein said reversibly immortalized mammalian liver cell line is CYNK-1 (deposited with International Patent Organism Depository, National Institute of Advanced Industrial Science and Technology, address: AIST Tsukuba Central 6, 1-1, Higashi 1-Chome, Tsukuba-shi, Ibaraki-ken, 305-8566 Japan, deposited date: Mar. 10, 2004, accession number: FERM BP-08657).

5. A mammalian liver cell obtainable by excising the immortalizing gene from a reversibly immortalized mammalian liver cell line or a passage cell line thereof of claim 1.

6. A bioartificial liver, comprising a reversibly immortalized mammalian liver cell line or a passage cell line thereof of claim 1.

7. A cell preparation, comprising as an active ingredient, a reversibly immortalized mammalian liver cell line or a passage cell line thereof of claim 1.

8. A non-viral vector, comprising a non-vial promoter and encoding an immortalizing gene between a pair of site-specific recombination sequences and a suicide gene in the outside of the pair of site-specific recombination sequences.

9. A bioartificial liver, comprising a mammalian liver cell of claim 5.

10. A cell preparation, comprising as an active ingredient, a mammalian liver cell of claim 5.
